(19) United States
(12) Patent Application Publication
(10) Pub. No.: US 2024/0343573 A1
(43) Pub. Date: Oct. 17, 2024

(54) GAS TREATMENT APPARATUS AND GAS TREATMENT METHOD

(71) Applicant: CANON KABUSHIKI KAISHA, Tokyo (JP)

(72) Inventors: KENJI TAKASHIMA, Kanagawa (JP); TAKUMI FURUKAWA, Shizuoka (JP); KAZUHIRO YAMAUCHI, Shizuoka (JP); MASAKI OZAWA, Shizuoka (JP); MOTOTERU GOTO, Shizuoka (JP); SHOTA KANEKO, Tokyo (JP)

(21) Appl. No.: 18/751,487

(22) Filed: Jun. 24, 2024

Related U.S. Application Data
(63) Continuation of application No. PCT/JP2022/047987, filed on Dec. 26, 2022.

(30) Foreign Application Priority Data
Dec. 28, 2021 (JP) 2021-215342

(51) Int. Cl. C01B 13/02; C25B 1/02

(52) U.S. Cl. CPC C01B 13/0203; C25B 1/02

(57) ABSTRACT

Provided is a gas treatment apparatus comprising an active oxygen supply device and a gas flow path, wherein the gas treatment apparatus is characterized in that the active oxygen supply device includes a housing having at least one opening, and a predetermined plasma actuator and a predetermined ozone decomposition device inside the housing, the plasma actuator and the ozone decomposition device are disposed so that an induced flow containing active oxygen flows out of the housing through the opening, and the active oxygen supply device is disposed so as to allow the induced flow containing the active oxygen flowing out through the opening to be introduced into the gas flow path.

### CROSS-REFERENCE TO RELATED APPLICATION

This is a continuation of International Application No. PCT/JP2022/047987, filed on Dec. 26, 2022, and designated the U.S., and claims priority from Japanese Patent Application No. 2021-215342 filed on Dec. 28, 2021, the entire contents of which are incorporated herein by reference.

### BACKGROUND OF THE INVENTION

### Field of the Invention

The present disclosure relates to a gas treatment device and a gas treatment method using active oxygen.

### Description of the Related Art

Japanese Patent Application Laid-open No. H06-335518 discloses a nascent oxygen generation device including: an apparatus body that is installed in a place where air flows, formed in a cylindrical shape in which a part of the air passes through, and has an inner surface made of metal with high ultraviolet reflectance; an ultraviolet lamp that is disposed on a shaft inside of the apparatus body and irradiates ultraviolet rays to decompose ozone; and an ozone generator that is provided on the upstream side of an airflow inside of the device body and converts oxygen in the air, which has been introduced into the body, into ozone by discharge. In paragraph [0016] of Japanese Patent Application Laid-open No. H06-335518, it is described that ozone generation performance and ozone decomposition performance are improved according to the nascent oxygen generation device, whereby large amounts of nascent oxygen from ozone are generated, and the generated nascent oxygen spreads into a refrigerator to oxidize and decompose bad-smelling substances inside of the refrigerator to deodorize the same.

### SUMMARY OF THE INVENTION

In the nascent oxygen generation device according to Japanese Patent Application Laid-open No. H06-335518, air introduced into the device from the outside comes into contact with active oxygen. Therefore, it is presumed that the nascent oxygen generation device could be used for treatment such as deodorization and sterilization of air outside the device. Therefore, the present inventors considered applying the nascent oxygen generation device according to Japanese Patent Application Laid-open No. H06-335518 to a gas treatment device. However, the performance of deodorization or sterilization of gas by the nascent oxygen generation device is restrictive.

At least an aspect of the present disclosure is aimed at providing a gas treatment device and a gas treatment method capable of more effectively treating gas using oxidative active oxygen.

At least an aspect of the present disclosure provides a gas treatment device comprising:
an active oxygen supply device; and
a gas flow path, wherein
the active oxygen supply device comprises
   a housing having at least one opening part,
   a plasma actuator arranged inside of the housing, and
   an ozone decomposition device,
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the ozone decomposition device decomposes the ozone contained in the induced flow to generate active oxygen in the induced flow, and the induced flow results in an induced flow containing the active oxygen,
the plasma actuator and the ozone decomposition device are arranged so that the induced flow containing the active oxygen flows to an outside of the housing from the opening part, and
the active oxygen supply device is arranged so that the induced flow containing the active oxygen is supplied to the gas flow path from the opening part.

At least an aspect of the present disclosure provides a treatment method for treating gas using active oxygen, the method comprising:
preparing a gas treatment device comprising an active oxygen supply device and a gas flow path, wherein
the active oxygen supply device comprises
   a housing having at least one opening part,
   a plasma actuator arranged inside of the housing, and
   an ozone decomposition device,
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the ozone decomposition device decomposes the ozone contained in the induced flow to generate active oxygen in the induced flows, and the induced flow results in an induced flow containing the active oxygen, and
the plasma actuator and the ozone decomposition device are arranged so that the induced flow containing the active oxygen flows to an outside of the housing from the opening part, and
the active oxygen supply device is arranged so that the induced flow containing the active oxygen is supplied to the gas flow path from the opening part.

At least an aspect of the present disclosure enables the provision of a gas treatment device capable of more effectively treating gas. Further, at least an aspect of the present disclosure enables the provision of a gas treatment method capable of more effectively treating gas.

Further features of the present invention will become apparent from the following description of exemplary embodiments with reference to the attached drawings.

### BRIEF DESCRIPTION OF THE DRAWINGS

FIGS. 1A and 1B are schematic cross-sectional views showing the configuration of a gas treatment device according to an aspect of the present disclosure;

FIGS. 2A and 2B are schematic cross-sectional views showing the configuration of a plasma actuator according to an aspect of the present disclosure;

FIG. 3 is an explanatory view of the plasma actuator according to an aspect of the present disclosure;

FIGS. 4A and 4B are schematic views showing the relationship between a first electrode and a second electrode;

FIGS. 5A to 5C are schematic views of doughnut-shaped electrodes;

FIGS. 6A and 6B are schematic cross-sectional views of a treatment test using the gas treatment device according to an aspect of the present disclosure; and

FIG. 7 is an explanatory view of a vector representing the blowing-out direction of an induced flow and a vector representing the flowing direction of gas.

### DESCRIPTION OF THE EMBODIMENTS

In the present disclosure, statements of “from XX to YY” and “XX to YY” each representing a numerical value range mean numerical value ranges including lower limits and upper limits, which are endpoints, unless otherwise particularly specified.

When numerical value ranges are stepwise stated, the upper and lower limits of the individual numerical value ranges can optionally be combined. In addition, in the present disclosure, such a statement as, e.g., “at least one selected from the group consisting of XX, YY, and ZZ” means any of XX, YY, ZZ, a combination of XX and YY, a combination of XX and ZZ, a combination of YY and ZZ, and a combination of XX, YY, and ZZ.

Further, “funguses” as targets for “sterilization” according to the present disclosure represent microorganisms, and the microorganisms include, in addition to true funguses, bacteria, single-celled algae, viruses, protozoans, or the like, animal or plant cells (including stem cells, dedifferentiated cells, and differentiated cells), tissue cultures, fused cells (including hybridoma) obtained by gene engineering, dedifferentiated cells, and transformants (microorganisms). Examples of viruses include noroviruses, rotaviruses, influenza viruses, adenoviruses, coronaviruses, measles viruses, rubella viruses, hepatitis viruses, herpes viruses, HIV, or the like. Further, examples of bacteria include Staphylococcus bacteria, E. coli bacteria, Salmonella, Pseudomonas aeruginosa, cholera bacteria, dysentery bacilli, Bacillus anthracis, tubercle bacilli, botulinum, tetanus bacilli, Streptococcus, or the like. Moreover, examples of true funguses include Trichophyton, Aspergillus, Candida, or the like. Accordingly, the “sterilization” according to the present disclosure includes, for example, the deactivation of viruses.

Moreover, active oxygen in the present disclosure includes, for example, free radicals such as superoxide (·O₂⁻) and hydroxy radicals (·OH) produced by the decomposition of ozone (O₃).

Hereinafter, embodiments for carrying out the present disclosure will be specifically illustrated with reference to the drawings. However, the dimensions, materials, shapes, their relative arrangements, or the like of constituting components described in the embodiments shall be appropriately modified depending on the configurations or various conditions of members to which the disclosure is applied. That is, the range of the disclosure does not intend to be limited to the following embodiments.

Moreover, configurations having the same functions will be denoted by the same symbols in the drawings, and their descriptions will be omitted depending on circumstances below.

According to studies by the present inventors, it is presumed that one reason for a restrictive gas treatment effect obtained by a nascent oxygen generation device according to Japanese Patent Application Laid-open No. H06-335518 is as follows.

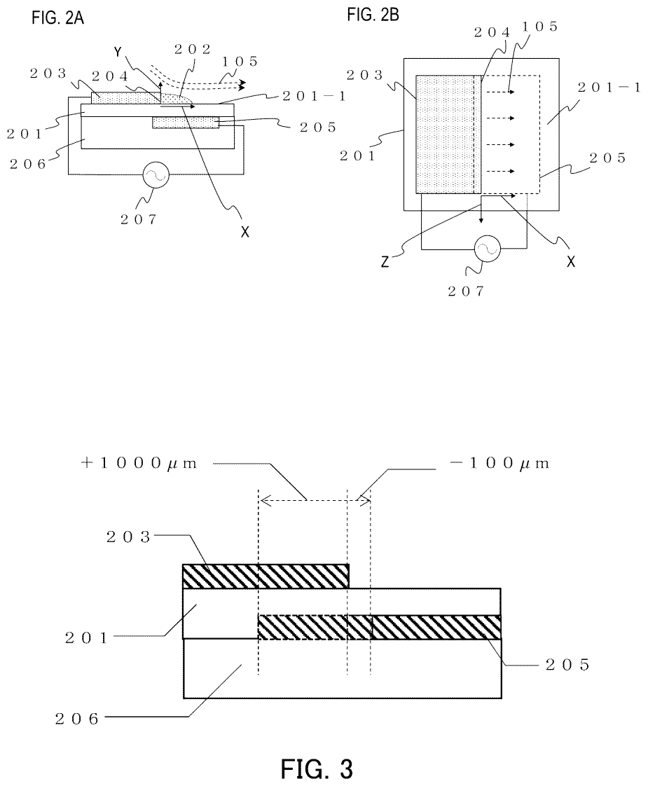

That is, active oxygen is highly unstable, extremely short with a half-life of 10⁻⁶ seconds for ·O₂⁻ and a half-life of 10⁻⁹ seconds for ·OH, and promptly converted into stable oxygen and water. Particularly, the nascent oxygen generation device according to Japanese Patent Application Laid-open No. H06-335518 is installed at a place where airflow is generated, and a part of air passes through the inside of a cylindrical device body. Specifically, it is disclosed in FIG. 2 of Japanese Patent Application Laid-open No. H06-335518 that an airflow is generated by a refrigerating machine fan 4 installed inside of a refrigerator. Even where active oxygen is generated inside of the device body in such a situation, it is presumed that the active oxygen is promptly converted into oxygen and water within the turbulence of air flowing in from the outside, and a contact opportunity between the active oxygen and odor substances and sterilization targets in the air reduces.

By such considerations, the present inventors have made further studies for the purpose of achieving a gas treatment device capable of more reliably using active oxygen for gas treatment to more effectively treat gas. As a result, the present inventors have found that a gas treatment device, with specific aspects that will be described below, contributes to the achievement of the object. Note that the gas treatment device according to the present disclosure will not be limited to the specific aspects below.

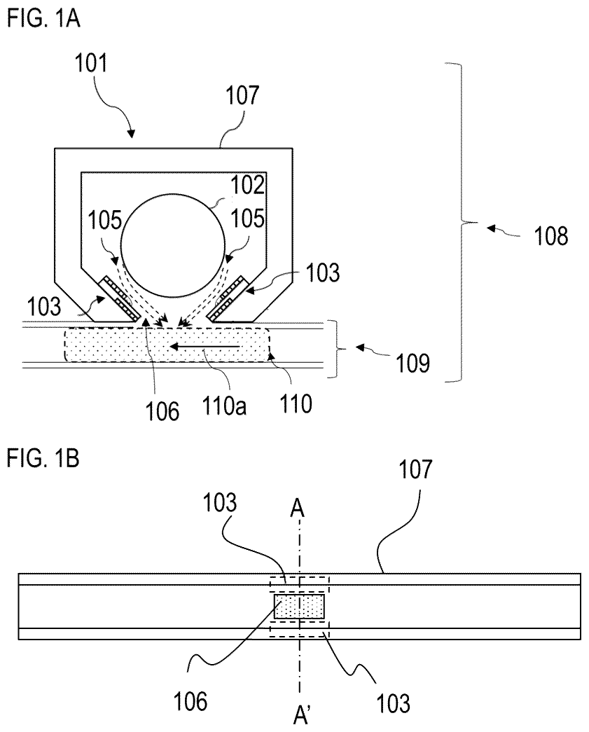

Hereinafter, a gas treatment device 108 according to an aspect of the present disclosure will be described using FIGS. 1A and 1B. The gas treatment device 108 according to the aspect of the present disclosure comprises an active oxygen supply device 101 and a gas flow path 109.

The active oxygen supply device 101 comprises a housing 107 having at least one opening part 106, and comprises an ultraviolet light source 102, which serves as an ozone decomposition device, and a plasma actuator (plasma generation device) 103 inside of the housing.

The ultraviolet light source 102 serving as the ozone decomposition device irradiates an induced flow 105 with ultraviolet rays to generate active oxygen in the induced flow 105. In FIG. 1A, symbol 110 denotes gas representing an object to be treated.

The gas flow path 109 represents a flow path for the gas 110 representing an object to be treated.

An aspect of the cross-sectional structure of the plasma actuator 103 is shown in FIGS. 2A and 2B. The plasma actuator is a so-called dielectric barrier discharge (DBD) plasma actuator (that will be simply called “DBD-PA” below depending on circumstances below) in which an exposed electrode (that will also be called a “first electrode” below) 203 with its end surface exposed is provided on one surface (that will also be called a “first surface” below) of a dielectric 201, and in which a second electrode 205 is provided on the surface (that will also be called a “second surface” below) on the side opposite to the first surface. In FIGS. 2A and 2B, symbol 206 denotes a dielectric substrate to embed the second electrode 205 in the plasma actuator in the thickness direction to prevent the generation of an induced flow from the end surface of the second electrode. Further, the application of a voltage to both the first electrode and the second electrode is possible with a power supply 207.

In the plasma actuator 103, the first electrode 203 and the second electrode 205, which are arranged via the dielectric 201, are arranged diagonally opposite to each other. By the application of a voltage between these electrodes (both the electrodes) from the power supply 207, a dielectric barrier discharge oriented from the first electrode 203 toward the second electrode 205 is generated. Then, plasma 202 is generated along an exposed part (a part not coated with the first electrode) 201-1 of the first surface of the dielectric 201 from an edge part 204 of the first electrode 203 toward the extending direction (an X-direction in FIGS. 2A and 2B) of the second electrode.

At the same time, an air-sucking flow oriented from the internal space of a container toward to the electrodes is also generated. Electrons in the surface plasma 202 collide with oxygen molecules in the air, and dissociate the oxygen molecules to generate oxygen atoms. After the generated oxygen atoms collide with undissociated oxygen molecules, ozone is generated. Accordingly, an induced flow 105 containing the ozone is generated along the surface of the dielectric 201 from the edge part 204 of the first electrode 203 by the operation of the jet-like flow by the surface plasma 202 and the air-sucking flow.

Further, the plasma actuator 103 and the ozone decomposition device 102 are arranged so that the induced flow 105 flows to the outside of the housing 107 from the opening part 106.

That is, the plasma actuator comprises the first electrode 203, the dielectric 201, and the second electrode 205 laminated together in this order, and the first electrode 203 is an exposed electrode provided on the first surface of the dielectric 201. Then, when a voltage is applied between the first electrode and the second electrode, the plasma actuator generates a dielectric barrier discharge oriented from the first electrode 203 toward the second electrode 205, and blows out an induced flow in a first direction (an X-direction in FIGS. 2A and 2B) representing one direction along the first surface of the dielectric 201 from the first electrode 203.

More specifically, the plasma actuator generates a dielectric barrier discharge oriented from the edge part 204 on one side of the first electrode 203 toward the second electrode 205, and blows out an induced flow representing a unidirectional jet flow in the first direction (a direction indicated by arrows 105 in FIGS. 2A and 2B) along the first surface of the dielectric 201 from the edge part 204 on the one side of the first electrode 203.

Further, the second electrode 205 extends in the blowing-out direction (first direction) of an induced flow in one cross section of the plasma actuator in the thickness direction.

More specifically, for example, the plasma actuator comprises the dielectric 201. When the cross section of the plasma actuator in the thickness direction is viewed, the first electrode 203 and the second electrode 205 are arranged diagonally opposite to each other via the dielectric 201 in the thickness direction of the plasma actuator. The first electrode 203 is provided to coat a part of the first surface of the dielectric 201, and the first surface of the dielectric has the exposed part 201-1 not coated with the first electrode 203.

FIG. 2B is a view of the plasma actuator when viewed through from the side of one surface of the dielectric. At least a part of the exposed part 201-1 and the second electrode 205 indicated by a broken line overlap each other. Accordingly, the overlap represents the region formed by the upper, lower, and right sides of the broken line indicating the second electrode 205 and the edge part 204 in FIG. 2B.

By the application of a voltage between the first electrode and the second electrode, an induced flow containing ozone is generated along the exposed part of the dielectric overlapping the second electrode 205 in the cross section (FIG. 2A) in the thickness direction, originating from the edge part 204 of the first electrode 203 on the side of the first direction.

Note that in the present disclosure, the first direction along the surface of the dielectric representing the blowing-out direction of an induced flow as shown in FIGS. 2A and 2B will be defined as an +X-direction depending on circumstances. Further, an axis including the +X-direction will be defined as an X-axis. For example, an X-axis direction collectively includes the +X-direction and the direction (an −X-direction) opposite to the +X-direction. The axis perpendicular to the X-axis and perpendicular to the first surface of the dielectric will be defined as a Y-axis. In the Y-axis, the direction of the first electrode when viewed from the dielectric (for example, a substantially vertically upward direction when the dielectric is set in a horizontal position) will be defined as a Y-direction. The direction perpendicular to the X-direction and along the first surface of the dielectric, that is, an axis perpendicular to the X-axis and Y-axis will be defined as a Z-axis.

An induced flow transforms into, for example, a wall-surface jet flow along the exposed part 201-1, and easily supplies ozone to a specific position. The length of the exposed part 201-1 in the direction of an induced flow (that is, the length from the edge part 204 of the first electrode on the side of the first direction to the edge part of the first surface of the dielectric) is not particularly limited but is preferably in the range from 0.1 mm to 50 mm, more preferably in the range from 0.5 mm to 20 mm, and still more preferably in the range from 1.0 mm to 10 mm.

In the active oxygen supply device according to an aspect of the present disclosure, the induced flow 105 containing ozone from the plasma actuator 103 flows to the outside of the housing 107 from the opening part 106 and is supplied to the gas flow path 109, while the ozone decomposition device 102 decomposes the ozone (for example, the ultraviolet light source 102 irradiates the induced flow 105 with ultraviolet rays) to generate active oxygen in the induced flow 105, thus enabling the active supply of the active oxygen to the gas flow path 109.

Therefore, before the generated active oxygen is converted into oxygen and water, it is possible to supply the active oxygen to the gas flow path 109 and treat gas representing an object to be treated.

As a result, the gas 110 is reliably treated by the active oxygen.

### Electrodes and Dielectric

Materials constituting the first electrode and the second electrode are not particularly limited as long as they have excellent conductivity. For example, metals such as copper, aluminum, stainless steel, gold, silver, and platinum and their plated or deposited materials, conductive carbon materials such as carbon black, graphite, and carbon nanotubes and their composite materials mixed with resins, or the like are usable. The material constituting the first electrode and the material constituting the second electrode may be the same or different from each other.

Among these materials, aluminum, stainless steel, or silver is preferable as the material constituting the first electrode from the viewpoint of avoiding the corrosion of the electrode and achieving uniform discharge. For the same reason, aluminum, stainless steel, or silver is preferable as the material constituting the second electrode.

Further, the first electrode and the second electrode may take the form of a flat plate, wire, needle, or the like without any restriction. The first electrode preferably takes the form of a flat plate. Further, the second electrode preferably takes the form of a flat plate. In a case where at least one of the first electrode and the second electrode takes the form of a flat plate, the flat plate preferably has an aspect ratio (the length of the long side/the length of the short side) of at least 2.

A material constituting the dielectric is not particularly limited as long as it has high electric insulating performance. Resins such as polyimide, polyester, fluorocarbon resin, silicone resin, acrylic resin, and phenol resin, glass, ceramics, and their composite materials mixed with resins, or the like are, for example, usable. Among these materials, the dielectric is preferably made of ceramics, glass, or polyimide. From the viewpoint of strength and insulating performance, ceramics or silicone resin is suitably used. Particularly, silicone resin is capable of enhancing the freedom of degree in the shape of the plasma actuator due to its flexibility.

In the plasma actuator, plasma is more easily generated as the shortest distance between the first electrode and the second electrode is smaller. Therefore, the thickness of the dielectric 201 present between the first electrode 203 and the second electrode 205 is preferably thin within the range where electric insulation breaks down does not occur when a voltage is applied between both the electrodes. Specifically, for example, in a case where the applied voltage is an AC voltage, the thickness of the dielectric portion may be in the range from 10 μm to 1,000 μm and preferably in the range from 10 μm to 250 μm when the difference between the maximum and minimum voltages of the AC voltage is in the range from 0.1 kVpp to 100 kVpp. Further, the shortest distance between the first electrode and the second electrode is preferably not more than 250 μm.

### Plasma Actuator

The plasma actuator is not particularly limited as long as the first electrode and the second electrode are provided via the dielectric and an induced flow representing a unidirectional jet flow containing ozone is capable of being generated by the application of a voltage between both the electrodes.

In the plasma actuator, plasma is more easily generated as the shortest distance between the first electrode and the second electrode is smaller. Therefore, the thinner the film thickness of the dielectric within the range where electric insulation breaks down does not occur, the more preferable it is. The film thickness may be in the range from 10 μm to 1,000 μm and preferably in the range from 10 μm to 200 μm. Further, the shortest distance between the first electrode and the second electrode is preferably not more than 200 μm.

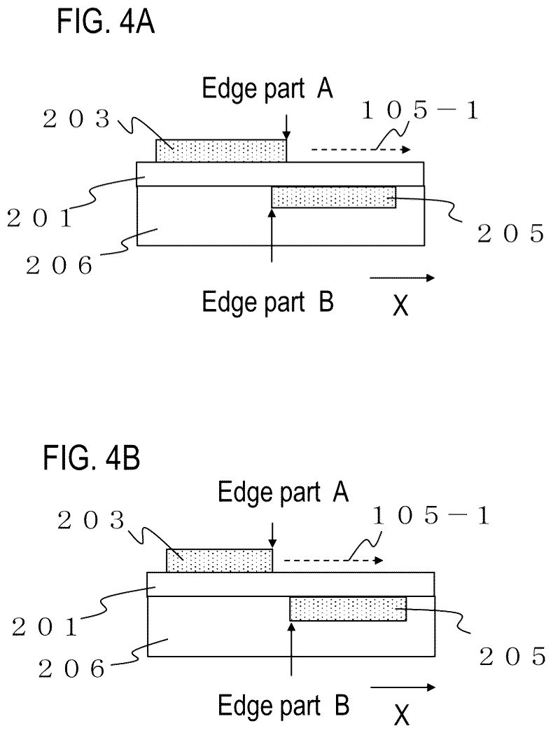

FIGS. 3, 4A, and 4B are explanatory views of the overlap between the first electrode 203 and the second electrode 205 of the plasma actuator serving as an ozone generation device. FIGS. 3, 4A, and 4B are cross-sectional views of the plasma actuator.

When the plasma actuator is viewed through from the side of the first electrode (first surface), the first electrode 203 and the second electrode 205 arranged diagonally opposite to each other are such that the edge part of the first electrode may be present in the formed area of the second electrode via the dielectric. For example, the first electrode and the second electrode may be provided to overlap each other in the Y-axis direction via the dielectric. In this case, it is preferable to prevent insulation from breaking down in the area, where the first electrode and the second electrode overlap each other via the dielectric, during the application of a voltage.

FIG. 4A shows an aspect in which the first electrode and the second electrode overlap each other (in the Y-axis direction) via the dielectric. In the cross section of the plasma actuator in the thickness direction, it is assumed that the edge part of the first electrode on the side of the first direction is an edge part A, and that the edge part of the second electrode on the side of a second direction (opposite to the X-direction) opposite to the first direction is an edge part B. At this time, the edge part B is preferably positioned on the side of the second direction (opposite to the X-direction) relative to the edge part A.

Further, assuming that the edge part of the first electrode on the side of the first direction is an edge part A and the edge part of the second electrode on the side of the second direction (opposite to the X-direction) opposite to the first direction is an edge part B when the plasma actuator is viewed through from the side of the first electrode, the edge part B is preferably positioned on the side of the second direction (opposite to the X-direction) relative to the edge part A.

The first electrode and the second electrode overlap each other via the dielectric as described above, thus enabling stable occurrence of plasma and an induced flow.

Further, since the first electrode and the second electrode are arranged diagonally opposite to each other via the dielectric 201, the edge part B is positioned on the side of the first direction (X-direction) relative to the edge part of the first electrode on the side opposite to the edge part A. Thus, the occurrence of an induced flow from the edge part of the first electrode on the side opposite to the edge part A is suppressed.

FIG. 4B shows an aspect in which the first electrode and the second electrode do not overlap each other (in the Y-axis direction) via the dielectric. Assuming that the edge part of the first electrode on the side of the first direction is an edge part A and the edge part of the second electrode on the side of the second direction (opposite to the X-direction) opposite to the first direction is an edge part B in the cross section of the plasma actuator in the thickness direction, the edge part B is preferably positioned on the side of the first direction (the X-direction) relative to the edge part A.

Further, assuming that the edge part of the first electrode on the side of the first direction is an edge part A and the edge part of the second electrode on the side of the second direction (opposite to the X-direction) opposite to the first direction is an edge part B when the plasma actuator is viewed through from the side of the first electrode, the edge part B is preferably positioned on the side of the first direction (the X-direction) relative to the edge part A. In a case where the first electrode and the second electrode do not overlap each other via the dielectric as described above, it is preferable to relatively increase the voltage applied between both the electrodes in order to compensate for the weakening of an electric field caused when the shortest distance between the electrodes relatively becomes large.

Further, assuming that the edge part of the first electrode on the side of the first direction is an edge part A and the edge part of the second electrode on the side of the second direction (opposite to the X-direction) opposite to the first direction is an edge part B when the plasma actuator is viewed through from the side of the first electrode, the edge part A and the edge part B preferably align with each other in the thickness direction (the Y-axis direction) of the dielectric as another aspect. Further, the edge part A and the edge part B preferably align with each other in the thickness direction (the Y-axis direction) of the dielectric in the cross section of the plasma actuator in the thickness direction as another aspect. This aspect refers to, for example, an aspect in which the edge part A and the edge part B are opposed to each other at the shortest distance via the dielectric, and the first electrode and the second electrode either do not overlap each other via the dielectric or do not separate from each other. Thus, the energy applied between both the electrodes is efficiently used to generate an induced flow.

Assuming that an overlapping length is considered positive, the overlap between the edge part A of the first electrode and the edge part B of the second electrode is preferably in the range from −100 μm to +1,000 μm, more preferably in the range from 0 μm to +200 μm, and still more preferably at 0 μm in the X-axis direction when viewed from the top of the cross-sectional view (FIG. 3). That is, assuming that a case where the edge part B is positioned on the side opposite to the blowing-out direction of an induced flow relative to the edge part A is considered positive, the interval between the edge part A and the edge part B in the direction (the X-axis direction) along the surface of the dielectric is preferably in the range from −100 μm to +1,000 μm, more preferably in the range from 0 μm to +200 μm, and still more preferably at 0 μm. However, it is difficult to achieve a constant overlap of 0 μm over the Z-axis direction from the viewpoint of machining accuracy in manufacturing the plasma actuator. Accordingly, it is common practice to provide a positive overlap corresponding to machining accuracy.

The thicknesses of both the first electrode and the second electrode are not particularly limited but may be in the range from 10 μm to 1,000 μm. When the thicknesses of the electrodes are at least 10 μm, the resistances of the electrodes become low, making it easier for plasma to be generated. When the thicknesses of the electrodes are not more than 1,000 μm, electric field concentration occurs more easily, making it easier for plasma to be generated.

The widths of both the first electrode and the second electrode (in the x-direction) are not particularly limited but may be at least 1,000 μm.

The shapes of the electrodes are not particularly limited but are preferably, for example, a rectangle such as an oblong and a square. The electrodes having a rectangle shape enable the generation of a uniform induced flow.

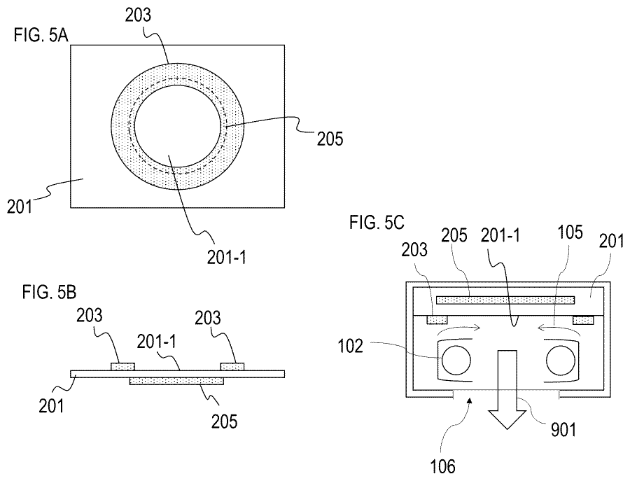

Further, as shown in FIGS. 5A (a perspective view from the side of the first surface of the dielectric) and 5B (a cross-sectional view of the plasma actuator in the thickness direction), the first electrode may have a doughnut shape, and the second electrode may have a circular or doughnut shape. Even with such electrode shapes, the first electrode 203 and the second electrode 205 are arranged diagonally opposite to each other via the dielectric 201 in the thickness direction of the plasma actuator when viewed in cross section in the thickness direction. Further, the first electrode 203 is provided to coat a part of the first surface of the dielectric 201, and the first surface has the exposed part 201-1 not coated with the first electrode 203. Moreover, when the plasma actuator is viewed through from the side of the first surface (FIG. 5A), at least a part of the exposed part 201-1 of the dielectric and the second electrode 205 overlap each other (at a hole in the doughnut-shaped first electrode).

Even in such a doughnut-shaped electrode DBD-PA, the application of a voltage between the first electrode and the second electrode results in the generation of a dielectric barrier discharge oriented from the first electrode toward the second electrode. As a result, an induced flow blows out in one direction along the surface of the dielectric from the first electrode. The blown-out induced flow collides near the center of the electrode and transforms into an axis-symmetric jet flow (a three-dimensional Wall normal jet) ejected upward in FIG. 5B.

An example of an active oxygen supply device using such a doughnut-shaped electrode DBD-PA is shown in FIG. 5C. In the active oxygen supply device shown in FIG. 5C, the induced flow 105 containing ozone is ejected from the edge part on the side of the inner periphery of the doughnut-shaped first electrode toward the first direction representing one direction along the surface of the dielectric 201, that is, toward a central direction. Then, the induced flow 105 collides at the central part of the first electrode, thus generating an axis-symmetric jet flow 901 containing the ozone toward the direction perpendicular to the surface (exposed part) 201-1 of the dielectric 201 (toward a vertically lower side in FIG. 5C). Then, the axis-symmetric jet flow 901 is excited by the ultraviolet light source 102 to decompose the ozone contained in the axis-symmetric jet flow into active oxygen, and the axis-symmetric jet flow containing the active oxygen flows to the outside of the housing 107 from the opening part 106 to be introduced into the gas flow path. Then, the gas 110 passing through the gas flow path is treated by the active oxygen contained in the axis-symmetric jet flow 901 flowing out from the opening part.

Note that as shown in FIG. 5C, the active oxygen supply device according to the present disclosure may be arranged inside of the housing so that the ozone decomposition device does not directly excite gas inside of the gas flow path 109 provided in the gas treatment device. In such an active oxygen supply device, active oxygen is hardly generated in situ in gas inside of the gas flow path unlike a case using the active oxygen supply device shown in FIG. 1A. However, due to an induced flow containing active oxygen flowing out from the opening of the housing, the active oxygen is actively supplied to the gas 110 inside of the gas flow path, thereby making it possible to reliably treat the gas 110.

Further, the plasma actuator may also be a so-called three-electrode plasma actuator in which a third electrode is additionally provided on a downstream side in the blowing-out direction of an induced flow from the first electrode and on the first surface of the dielectric 201. In this case, for example, it is possible to apply an AC voltage using the first electrode as an AC electrode, and apply a DC voltage using the third electrode as a DC electrode. It is also possible to generate a sliding discharge by applying a negative DC voltage to the DC electrode.

Further, in a case where the edge part of the second electrode is exposed, plasma may be generated also from the edge part of the second electrode, potentially resulting in the generation of an induced flow in the direction opposite to the induced flow 105 derived from the first electrode. In the gas treatment device according to this aspect, it is preferable to minimize ozone concentration in the inner space of the gas treatment device, excluding a region where gas representing an object to be treated is treated, as much as possible. Further, it is preferable to prevent the generation of an airflow that disrupts the induced flow 105 in a container. Therefore, it is preferable to prevent the generation of an induced flow derived from the second electrode.

In view of this, the second electrode 205 is preferably an embedded electrode so that plasma is not generated from the second electrode 205. For example, as shown in FIGS. 2A and 2B and FIG. 3, the second electrode may be coated with a dielectric such as the dielectric substrate 206, or embedded in the dielectric 201. The second electrode may be embedded to such a degree that the generation of plasma from the edge part of the second electrode is prevented. For example, a part of the surface of the second electrode may be exposed, and the exposed surface of the second electrode and the dielectric substrate 206 or the dielectric 201 may form the same plane. The edge part of the second electrode is preferably coated with the dielectric substrate 206 or the dielectric 201. Accordingly, the plasma actuator is preferably, for example, a single dielectric barrier discharge (SDBD) plasma actuator.

In the plasma actuator, an induced flow is preferably not generated from edge parts other than the edge part A of the first electrode defined as described above. Therefore, the edge parts other than the edge part A may be coated with the dielectric. In this manner, a unidirectional jet flow is generated even when the first electrode and the second electrode overlap each other in the Y-axis direction. Further, the shapes of the electrodes may be controlled so that an induced flow is not generated from the edge parts other than the edge part A according to the relationship between the first electrode and the second electrode. For example, in a case where the electrodes have the form of a rectangle, the first electrode and the second electrode may have the same length or the first electrode may be longer than the second electrode in the Z-axis direction (the direction perpendicular to the blowing-out direction of an induced flow from the edge part A). According to such an aspect, an induced flow is actively easily supplied to the gas flow path.

The induced flow 105 containing ozone flows in the flowing direction of a jet-like flow generated by surface plasma along the exposed part 201-1 of the first surface of the dielectric 201 from the edge part 204 of the first electrode 203, that is, in the direction along the exposed part 201-1 of the first surface of the dielectric from the edge part 204 of the first electrode 203. This induced flow is a flow of gas containing ozone with a speed of approximately several meters per second to several tens of meters per second.

The voltage applied between the first electrode 203 and the second electrode 205 of the plasma actuator is not particularly limited as long as it enables the plasma actuator to generate plasma. Further, a DC voltage or an AC voltage may be applied, but the AC voltage is preferably applied. Further, a pulse voltage may be preferably applied as the voltage as an aspect.

Moreover, the amplitude and frequency of the voltage may be appropriately set in order to adjust the flow velocity of an induced flow and ozone concentration in the induced flow. In this case, the amplitude and frequency of the voltage may be appropriately selected from the viewpoint of generating ozone concentration, which is necessary for generating an effective active oxygen concentration or effective active oxygen amount according to the purpose of treatment, in the induced flow, supplying generated active oxygen to gas representing an object to be treated while maintaining an effective active oxygen concentration or effective active oxygen amount according to the purpose of treatment, or the like.

For example, the difference between the maximum and minimum voltages of the AC voltage may be in the range from 1 kVpp to 100 kVpp. Furthermore, the frequency of the voltage may be preferably at least 1 kHz and more preferably in the range from 10 kHz to 100 kHz.

In a case where the voltage is an AC voltage, the waveform of the AC voltage is not particularly limited, and a sine wave, a rectangular wave, a triangular wave, or the like may be employed. However, the rectangular wave is preferable from the viewpoint of a fast rising voltage.

The duty ratio of the voltage is also appropriately selectable, but the voltage preferably rises fast. Preferably, the voltage is applied so that the rate of voltage rise, which reaches from the bottom to the top of the amplitude of a wavelength, becomes at least 10,000,000 V per second.

Note that a value obtained by dividing the amplitude of the voltage applied between the first electrode 203 and the second electrode 205 by the film thickness of the dielectric 201 (voltage/film thickness) is preferably at least 10 kV/mm.

Ozone Decomposition Device The active oxygen supply device comprises the ozone decomposition device 102. The ozone decomposition device decomposes ozone contained in an induced flow to generate active oxygen in the induced flow. An example of an ozone decomposition device includes one that is capable of acting on ozone contained in an induced flow and decomposing the ozone. The ozone decomposition device is preferably one that is capable of decomposing ozone without disrupting an induced flow.

The ozone decomposition device is preferably at least one device selected from a group consisting of an ultraviolet light source irradiating an induced flow with ultraviolet rays to generate active oxygen in the induced flow, a heating device heating an induced flow to generate active oxygen in the induced flow, and a humidifying device humidifying an induced flow to generate active oxygen in the induced flow. The ozone decomposition device may be a combination of these devices. For example, the ozone decomposition device may be a device that heats an induced flow while irradiating the induced flow with ultraviolet rays or a device that humidifies the inside of the housing while irradiating an induced flow with ultraviolet rays and heating the same. The ozone decomposition device is more preferably an ultraviolet light source. The respective devices will be described below.

### Ultraviolet Light Source and Ultraviolet Rays

The ultraviolet light source is not particularly limited as long as it is capable of exciting ozone and irradiating ultraviolet rays enabling the generation of active oxygen. Further, the ultraviolet light source is not particularly limited as long as it excites ozone and has the wavelength and illumination of ultraviolet rays necessary for obtaining an effective active oxygen concentration and an effective active oxygen amount according to the purpose of treatment.

For example, the peak value of the light absorption spectrum of ozone is 260 nm. From this point of view, the peak wavelength of the ultraviolet rays is preferably in the range from 220 nm to 310 nm, more preferably in the range from 253 nm to 285 nm, and still more preferably in the range from 253 nm to 266 nm.

As a specific ultraviolet light source, a low-pressure mercury lamp in which mercury is enclosed in quartz glass together with inactive gas such as argon and neon, a cold-cathode tube ultraviolet lamp (UV-CCL), an ultraviolet LED, or the like is usable. The wavelength of the low-pressure mercury lamp or the cold-cathode tube ultraviolet lamp may be selected from options such as 254 nm. On the other hand, the wavelength of the ultraviolet LED may be selected from options such as 265 nm, 275 nm, and 280 nm from the viewpoint of output performance.

### Heating Device

The heating device is not particularly limited as long as it is capable of exciting ozone in an induced flow and providing heat energy enabling the generation of active oxygen. The thermal decomposition of ozone starts at approximately 100° C. From this point of view, a device capable of heating an induced flow to approximately 120° C. is preferable. On the other hand, if the heating temperature of the heating device exceeds 120° C., there may be a case where thermal degradation such as melting and decomposition occurs in the housing or the gas flow path depending on the material of the housing or the gas flow path. Therefore, the heating temperature is preferably not more than 200° C.

The heating device is not particularly limited but a ceramic heater, a cartridge heater, a sheathed heater, an electric heater, an oil heater, or the like is usable. In the case of a device including a metal heating element, the heating element is preferably made of a material such as a nichrome alloy and tungsten having excellent oxidation resistance. A cartridge heater is preferably used.

### Humidifying Device

The humidifying device is not particularly limited as long as it is capable of humidifying the inside of the housing to cause water to be contained in an induced flow and decomposing ozone in the induced flow with the water to generate active oxygen in the induced flow. Here, the humidifying process involves giving moisture to a target, and the form of the moisture is not particularly limited but may be at least one selected from a group including gas, a liquid, and a solid. Further, known water is arbitrarily usable as water for giving moisture, and this water may contain substances other than water.

The humidifying device is not particularly limited but is, for example, a vaporizing type humidifying device or a mist type humidifying device.

In order to suppress humidity near the plasma actuator, the humidifying device is preferably one that has directivity with respect to a moisture supply direction (that will also be simply called directivity below). With the directivity of the humidifying device, it is possible to efficiently humidify the vicinity of an induced flow or the vicinity of the surface of an object to be treated without increasing humidity near the plasma actuator.

In order to make the humidifying device have the directivity, a known method is suitably available. For example, a method in which an airflow is generated by a fan and moisture is supplied to the direction of the airflow, a method in which an appropriate pressure is given to moisture by an air pump or the like to inject the moisture in a desired direction, or the like, is available. In order to prevent an induced flow from being disrupted, the humidifying device is preferably directed in the same direction (first direction) as the induced flow.

### Gas Flow Path

The gas treatment device 108 comprises the gas flow path 109. The gas flow path 109 is not particularly limited as long as the gas 110 representing an object to be treated is treated by the active oxygen supply device 101 in the process of flowing inside of the gas flow path. The size or shape of the gas flow path, the relative position of the gas flow path and the active oxygen supply device 101, a gas flow mechanism, or the like may be appropriately selected so that, for example, generated active oxygen is capable of being actively supplied to the gas flow path while maintaining an effective active oxygen concentration or effective active oxygen amount according to the purpose of treatment.

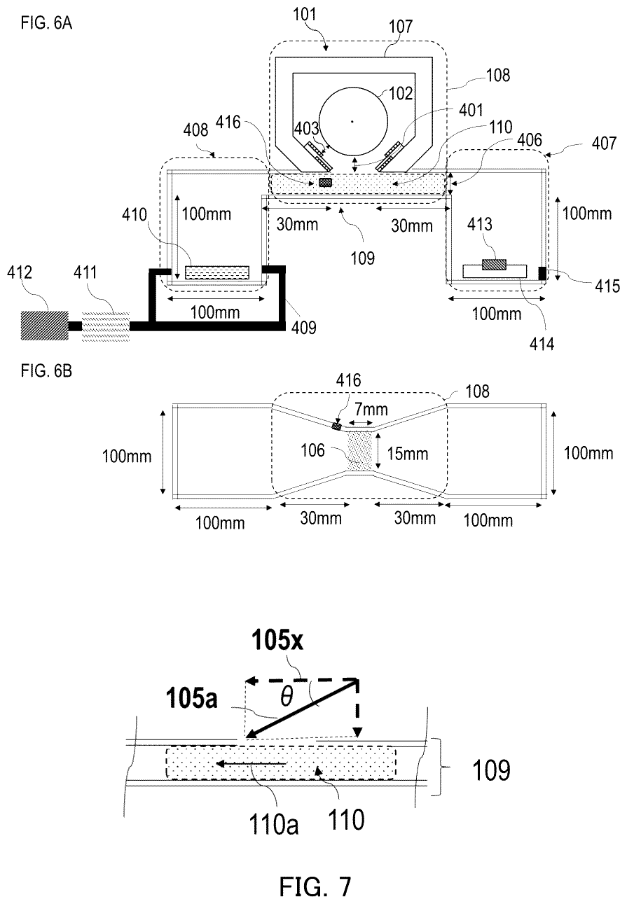

The width (denoted by symbol 406 in FIG. 6A) of the gas flow path 109 is not particularly limited but may be appropriately set. Here, the width of the gas flow path 109 refers to the length of the gas flow path in the direction perpendicular to a flat plate when the flat plate is abutted onto the opening part 106 of the active oxygen supply device.

For example, in order to exhibit sufficient sterilization performance, it is preferable to sufficiently supply generated active oxygen to the side of the gas flow path opposed to the active oxygen supply device 101. For this reason, the width of the gas flow path is preferably short. That is, the width of the gas flow path is preferably not more than 100 mm, more preferably not more than 50 mm, and still more preferably not more than 12 mm.

Further, the width of the gas flow path is only required to exceed 0 mm. However, from the viewpoint of increasing a gas treatment amount per unit time, the width of the gas flow path is preferably at least 3 mm and more preferably at least 6 mm.

The flow velocity of gas (that is, a gas flow velocity) inside of the gas flow path is not particularly limited, as long as the gas 110 representing an object to be treated is treated by the active oxygen supply device 101 in a space facing the opening part that is a spot where active oxygen in an induced flow supplied by the active oxygen supply device is believed to sufficiently act. From the viewpoint of sufficiently performing gas treatment, the flow velocity of gas is preferably not more than 2 m/s and more preferably not more than 0.8 m/s in the space facing the opening part. Further, the flow velocity is generally at least 0 m/s, more preferably at least 0.05 m/s, and still more preferably at least 0.2 m/s.

Here, the space facing the opening part refers to a space where a perpendicular line to the surface of the opening part passes through inside of the gas flow path.

### Housing and Opening Part

The active oxygen supply device comprises: the housing 107 having the at least one opening part 106; the ozone decomposition device 102 arranged inside of the housing; and the plasma actuator 103.

The opening part is not particularly limited as long as the induced flow 105 generated from the plasma actuator 103 flows to the outside of the housing 107. The size of the opening part, the position of the opening part, and the relative position of the opening part and an object to be treated may be appropriately selected so that, for example, generated active oxygen is capable of being actively supplied to the gas flow path while maintaining an effective active oxygen concentration or effective active oxygen amount according to the purpose of treatment.

### Arrangement of Plasma Actuator, Ozone Decomposition Device, and Gas Flow Path

In the active oxygen supply device 101, the position of the plasma actuator 103 that generates an induced flow containing ozone is not particularly limited as long as the plasma actuator 103 is arranged so that the induced flow 105 flows to the outside of the housing from the opening part and is supplied to the gas flow path while maintaining an effective active oxygen concentration or effective active oxygen amount according to the purpose of treatment due to ultraviolet rays irradiated from the ultraviolet light source 102 representing the ozone decomposition device. The same applies to a case where the ozone decomposition device is a heating device or a humidifying device. For example, the plasma actuator and the ozone decomposition device may be arranged so that the induced flow 105 containing generated active oxygen is supplied to the gas flow path at the shortest distance.

Further, for example, the extension line in the direction (same as the +X-direction) along the first surface of the dielectric from the edge part 204 of the first electrode 203 of the plasma actuator on the side of the first direction is preferably directed to the opening part. Thus, an induced flow easily flows to the outside of the housing from the opening part and is supplied to the gas flow path.

Moreover, in the gas treatment device 108 according to the present disclosure, an angle θ (that will also be called an induced flow incident angle) formed between a vector 105a representing the blowing-out direction of the induced flow 105 containing active oxygen, which flows to the outside of the housing 107 from the opening part 106, and a vector representing the flowing direction of the gas 110 as shown in FIG. 7 is not limited as long as the induced flow and the gas are mixed together. Accordingly, the induced flow incident angle θ is, for example, preferably more than 0° and not more than 90°. Particularly, as shown in FIG. 7, the vector 105a representing the blowing-out direction of the induced flow 105 containing active oxygen, which flows to the outside of the housing 107 from the opening part 106, preferably has a vector component 105x parallel to the direction of an arrow 110a indicating the vector representing the flowing direction of the gas 110. In this case, the induced flow incident angle θ is preferably in the range from more than 0° and less than 90° and more preferably in the range from 30° to 60°.

By establishing the above relationship between the blowing-out direction of the induced flow 105 from the opening part and the flowing direction of the gas 110, it is possible to greatly enhance the effect of gas treatment using active oxygen. The present inventors presume the reason for this enhancement as follows.

As described above, active oxygen is capable of maintaining its active state for a longer period of time inside of the straightened flow of an induced flow supplied from the active oxygen supply device according to the present disclosure. Meanwhile, the gas 110 flowing in the direction of the arrow 110a moves in the same direction as the arrow 110a. Therefore, since the vector 105a representing the blowing-out direction of an induced flow has the component 105x parallel to the direction of the arrow 110a, which is the flowing direction of the gas 110, the straightened airflow of the induced flow 105 supplied to the gas 110 is hardly disrupted by the gas 110, thus enabling the gas 110 to be placed under an environment where active oxygen exists for a longer period of time. As a result, it is presumed that the effect of gas treatment using active oxygen is enhanced.

The arrangement of the ozone decomposition device is not particularly limited as long as the ozone decomposition device is arranged to be capable of generating active oxygen in an induced flow and performing gas treatment while maintaining an effective active oxygen concentration or effective active oxygen amount according to the purpose of treatment.

As described above, an induced flow containing ozone is actively supplied to the gas flow path. Further, if the ozone decomposition device is an ultraviolet light source, it is possible to irradiate the induced flow with ultraviolet rays to generate active oxygen in the induced flow. Therefore, as the induced flow is irradiated with ultraviolet rays, the ozone is excited, thus enabling the active supply of the induced flow where the active oxygen has been generated to the gas flow path and a significant increase in an active oxygen concentration or active oxygen amount inside of the gas flow path.

On the other hand, in a case where the ozone decomposition device is arranged to be capable of treating gas passing through the gas flow path via the opening part, the ozone decomposition device is capable of decomposing undecomposed ozone present in an induced flow in situ in the presence region of gas representing an object to be treated and generating active oxygen. As a result, it is possible to further increase a treatment degree or treatment efficiency.

The relative position of the ozone decomposition device and the plasma actuator is not particularly limited as long as the ozone decomposition device and the plasma actuator are arranged to be capable of generating active oxygen in an induced flow and performing gas treatment while maintaining an effective active oxygen concentration and effective active oxygen amount according to the purpose of the treatment.

Further, since the distance between the ozone decomposition device and the plasma actuator varies depending on the purpose of treatment, it may not be stipulated definitively. For example, the distance between the opposing surface of the dielectric of the plasma actuator and the ozone decomposition device is preferably not more than 10 mm and more preferably not more than 4 mm. However, it is not necessary to arrange the plasma actuator at a place within approximately 10 mm from the ozone decomposition device. The distance between the ozone decomposition device and the plasma actuator is not particularly limited as long as active oxygen in an induced flow has an effective concentration according to the purpose of treatment in consideration of elements enabling the decomposition of ozone that will be described later, such as the illumination and wavelength of ultraviolet rays.

Further, it is also preferable to provide at least one of the ozone decomposition device and the plasma actuator with a moving unit and make at least one of the ozone decomposition device and the plasma actuator freely movable to enable a uniform ozone decomposition degree as an aspect.

In the relative position of the active oxygen supply device and the gas flow path, at least one of the active oxygen supply device and the gas flow path may be arranged so that active oxygen is generated in an induced flow and the induced flow maintaining an effective active oxygen concentration and effective active oxygen amount according to the purpose of treatment is supplied to the gas flow path.

Further, in a case where the ozone decomposition device is an ultraviolet light source, the ultraviolet light source may be arranged at a position where gas representing an object to be treated is capable of being irradiated with ultraviolet rays, or may be arranged at a position where gas representing an object to be treated is not capable of being irradiated with ultraviolet rays. Even in a case where gas representing an object to be treated is not capable of being irradiated with ultraviolet rays from the ultraviolet light source, the treatment device using active oxygen according to this aspect enables treatment by exposing the gas representing the object to be treated to the active oxygen in an induced flow.

Similarly, in a case where the ozone decomposition device is a heating device, the heating device may be arranged at a position where gas representing an object to be treated is capable of being heated, or may be arranged at a position where gas representing an object to be treated is not capable of being heated.

Moreover, in gas treatment using ultraviolet rays alone, a treatment target is only an area where the ultraviolet rays are irradiated. However, in gas treatment by the active oxygen supply device according to the prese disclosure, it is possible to treat gas present at a position where active oxygen could reach. Accordingly, it is possible to efficiently treat gas.

On the other hand, as described above, in a case where the ultraviolet light source is arranged to be capable of irradiating gas passing through the gas flow path with ultraviolet rays, the ultraviolet light source is capable of decomposing undecomposed ozone present in an induced flow in situ in the presence region of gas representing an object to be treated and generating active oxygen. As a result, it is possible to further increase a treatment degree or treatment efficiency.

In this case, the illumination of ultraviolet rays in the opening part is not particularly limited but is preferably set to be capable of decomposing ozone contained in an induced flow, generating active oxygen in the induced flow, and generating an effective active oxygen concentration or effective active oxygen amount according to the purpose of treatment even in, for example, the opening part. Specifically, the illumination of ultraviolet rays in the opening part is, for example, preferably at least 40 W/cm², more preferably at least 100 μW/cm², still more preferably at least 400 W/cm², and particularly preferably at least 1,000 μW/cm². An upper limit of the illumination is not particularly limited but may be, for example, not more than 10,000 μW/cm².

Further, since the distance between the ozone decomposition device and the opening part also varies depending on the purpose of treatment, it may not be stipulated definitively. For example, the distance is preferably not more than 10 mm and more preferably not more than 4 mm. However, it is not necessary to arrange the opening part at a place within approximately 10 mm from the ozone decomposition device. The distance between the ultraviolet light source and the opening part is not particularly limited as long as active oxygen in an induced flow has an effective concentration according to the purpose of treatment in consideration of the illumination of ultraviolet rays or the like.

Further, an ozone generation amount per unit time in a state in which ozone in an induced flow is not decomposed by the ozone decomposition device is, for example, preferably at least 15 μg/min in the plasma actuator. The ozone generation amount is more preferably at least 30 μg/min. An upper limit of the ozone generation amount is not particularly limited but is, for example, not more than 1,000 μg/min.

The flow velocity of an induced flow may be, for example, a velocity at which generated active oxygen is capable of being actively supplied to the gas flow path while maintaining an effective active oxygen concentration or effective active oxygen amount according to the purpose of treatment. The flow velocity is, for example, in the range from approximately 0.01 m/s to 100 m/s as described above.

The concentration of ozone in an induced flow generated from the plasma actuator as described above or the flow velocity of the induced flow may be controlled by the thicknesses or materials of the electrodes or the dielectric, the type, amplitude, frequency of an applied voltage, or the like.

The gas treatment device according to the present disclosure is applicable to various applications implemented by supplying active oxygen to gas representing an object to be treated. For example, the gas treatment device according to the present disclosure is applicable also to purposes such as sterilization and deodorization of gas.

Further, the present disclosure provides a treatment method for treating gas using active oxygen, the method comprising:
a step of preparing a gas treatment device comprising an active oxygen supply device and a gas flow path, wherein
the active oxygen supply device comprises
   a housing having a least one opening part,
   a plasma actuator arranged inside of the housing, and
   an ozone decomposition device,
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the ozone decomposition device decomposes the ozone contained in the induced flow to generate active oxygen in the induced flow, and the induced flow results in an induced flow containing the active oxygen, and
the plasma actuator and the ozone decomposition device are arranged so that the induced flow containing the active oxygen flows to the outside of the housing from the opening part, and
the active oxygen supply device is arranged so that the induced flow containing the active oxygen is supplied to the gas flow path from the opening part.

Note that the “effective active oxygen concentration or effective active oxygen amount” in the present disclosure refers to an active oxygen concentration or active oxygen amount for achieving purposes for an object to be treated, such as sterilization and deodorization, and may be appropriately adjusted according to purposes using the thicknesses and materials of the electrodes and the dielectric constituting the plasma actuator, the type, amplitude, and frequency of an applied voltage, elements enabling the decomposition of ozone such as the illumination and irradiation time of ultraviolet rays, an induced flow incident angle, or the like.

### EXAMPLES

The present disclosure will be described in further detail using Examples and Comparative Examples below, but the aspects of the present disclosure are not limited to the Examples and Comparative Examples.

### Example 1

### 1. Manufacturing of Active Oxygen Supply Device

An aluminum foil having a longitudinal length of 2.5 mm, a horizontal length of 15 mm, and a thickness of 100 μm was pasted onto a first surface of a glass plate (having a longitudinal length of 5 mm, a horizontal length (a depth direction in FIG. 1A) of 18 mm, and a thickness of 150 μm) serving as a dielectric, using an adhesive tape to form a first electrode. Further, an aluminum foil having a longitudinal length of 3 mm, a horizontal length of 15 mm, and a thickness of 100 μm was pasted onto a second surface of the glass plate using an adhesive tape so as to be diagonally opposite to the aluminum foil pasted onto the first surface to form a second electrode. Moreover, the second surface including the second electrode was coated with a polyimide tape. In this manner, a plasma actuator was manufactured with the first electrode and the second electrode overlapping each other over a width of 200 μm via the dielectric (glass plate). This plasma actuator was prepared in pairs.

Next, as a housing 107 of an active oxygen supply device 101, a case made of an ABS resin, having a height of 25 mm, a width of 20 mm, a length 170 mm, and a thickness of 2 mm, and having a substantially trapezoidal cross-sectional shape shown in FIG. 1A as a cross-sectional shape (a cross section cut out along the line A-A′ in FIG. 1B) in a short direction was prepared. FIG. 1B is a view of the case when viewed from an opening part 106. The case had the rectangular opening part 106 having a width of 7 mm and a length of 15 mm to be symmetrical about the center (A-A′ indicated by a one-dot dashed line in FIG. 1B) having a length of 170 mm in the longitudinal direction. Next, the two previously-manufactured plasma actuators 103 were fixed to the oblique-line portion of the housing 107 in FIG. 1A so that an induced flow incident angle θ became 45°. Further, at the attachment position of the plasma actuators 103 (a dashed-line portion in FIG. 1B) when viewed from the lower surface of the housing (FIG. 1B), the plasma actuators 103 were fixed so that the center line (A-A′ indicated by the one-dot dashed line) of the 170 mm length of the housing, the center line of the opening part, and the centers of the plasma actuators were aligned with each other.

Moreover, an ultraviolet lamp 102 (a cold-cathode tube ultraviolet lamp, product name: UW/9F89/9, manufactured by Stanley Electric Co., Ltd, cylindrical shape having a diameter of 9 mm and a peak wavelength of 254 nm) serving as an ozone decomposition device was arranged inside of the housing. The ultraviolet light source 102 was arranged so that the distance (denoted by symbol 403 in FIG. 6A) between the ultraviolet lamp 102 and exposed parts 201-1 of first surfaces of dielectrics 201 of the plasma actuators became 2 mm, and so that the distance (denoted by symbol 401 in FIG. 6A) between the ultraviolet light source and the surface of a flat plate on the side opposite to the ultraviolet light source became 3 mm when the flat plate was abutted onto the opening part 106 of the housing 107. In this manner, the active oxygen supply device according to this Example was manufactured.

At the position of the opening part 106 serving as a supply port for active oxygen in the active oxygen supply device 101, a spectroradiometer (product name: USR-45D, manufactured by Ushio Inc.) was placed to measure the illumination of ultraviolet rays. The measured illumination was 1370 μW/cm² based on the integrated value of the spectrum. At this time, the plasma actuators were not powered on to prevent an affect by the shielding of ultraviolet rays due to ozone generated from the plasma actuators.

Subsequently, the active oxygen supply device 101 was placed into a sealed container (not shown) having a volume of 1 L to calculate the amount of ozone generated from the plasma actuators 103. The sealed container was equipped with an opening sealable with a rubber plug, thus enabling the suction of internal gas from the opening using an injector. Then, a voltage with a sine waveform having an amplitude of 2.4 kVpp and a frequency of 80 kHz was applied to the plasma actuators 103. After 1 minute had elapsed, 100 ml of the gas inside of the sealed container was collected. The collected gas was sucked by an ozone detection tube (product name: 182SB, manufactured by KOMYO RIKAGAKU KOGYO K.K.) to measure the concentration (PPM) of measured ozone contained in an induced flow from the plasma actuators 103. Using the value of the measured ozone concentration, an ozone generation amount per unit time was calculated by the following formula.

$$Amount of ozone generated per unit time ( \frac{mg}{min} ) = Measured ozone concentration ( PPM ) * \frac{Molecular weight of ozone 48}{22.4} * \frac{\frac{273}{273 + Room temperature ( °C )}}{10000} * \frac{Gas inside of sealed container ( L )}{Collected gas ( L )} = Measured ozone concentration ( PPM ) * 48 / 22.4 * 273 / ( 273 + 25 ) / 10000 * 0.1 / 1$$

As a result, the ozone generation amount per unit time was 39 μg/min. At this time, the ultraviolet light source was not powered on to prevent an affect by ozone decomposition due to ultraviolet rays irradiated from the ultraviolet light source.

Finally, the ozone generation amount during the operation of both the plasma actuators 103 and the ultraviolet lamp 102 was measured. The operating condition of the plasma actuators 103 was such that 39 μg/min of ozone was generated when only the plasma actuators 103 were operated. Further, the operating condition of the ultraviolet lamp 102 was such that an illumination of 1370 μW/cm² was obtained when only the ultraviolet lamp 102 was operated. As a result, the ozone generation amount was 8 μg/min in a case where both the plasma actuators 103 and the ultraviolet lamp 102 were operated. It appears that 31 μg/min, which corresponds to the reduction amount from 39 μg/min, represents the amount of ozone transformed into active oxygen.

### 2. Manufacturing of Gas Treatment Device and Test Mechanism

A gas treatment device 108 used in this Example includes the active oxygen supply device 101 manufactured in the manner described above. With reference to FIGS. 6A and 6B, the configuration of a test mechanism using the gas treatment device according to this Example will be described.

Inside the gas treatment device, a gas flow path 109 other than the active oxygen supply device was manufactured using an acrylic member containing an acrylic resin.

A mechanism for an evaluation test that will be described later was manufactured concurrently. As shown in FIG. 6A, the test mechanism is composed of a fungus-containing-gas preparation unit 407 and a fungus collection unit 408, additionally the gas treatment device 108. The fungus-containing-gas preparation unit 407, the gas treatment device 108, and the fungus collection unit 408 were installed to be continuous in this order. The external surfaces of the fungus-containing-gas preparation unit 407 and the fungus collection unit 408 were also manufactured using an acrylic member containing an acrylic resin in the same manner as the gas flow path.

Holes having a diameter of 5 mm were provided on wall surfaces in four directions adjacent to the bottom surface of the fungus collection unit 408, and a water trap 411 and a suction pump 412 (mini pump MP-2N, manufactured by SHIBATA SCIENTIFIC TECHNOLOGY LTD., variable flow controlled by a flowmeter) were connected in this order via a resin tube 409. During the test, the suction pump was operated to circulate gas.

A distance 406 corresponding to the height of the gas flow path up to the wall surface opposed to the opening part 106 in the gas flow path 109 was set as shown in FIG. 6A. Note that the distance 406 was set at 6 mm.

The flow velocity of gas refers to the gas flow velocity in a space facing the opening part where active oxygen generated by the active oxygen supply device is believed to act sufficiently. Here, the space facing the opening part refers to the space where a perpendicular line to the surface of the opening part passes through inside of the gas flow path. For the measurement of the flow velocity of gas, an opening/closing window 416 was provided at a spot adjacent to the side of the fungus collection unit 408 with respect to the space facing the opening part, and a detection rod as a wind meter (an anemomaster wind velocity meter MODEL 6006, manufactured by KANOMAX JAPAN INC.) was inserted into the opening/closing window 416.

### 3. Evaluation

### 3-1. Confirmation Test for Active Oxygen

The presence or absence of active oxygen in an induced flow flowing out from the opening part was confirmed by making use of the decolorization of methylene blue (see “Magnetic Field Effect on the Photocatalytic Reaction with TiO₂ Semiconductor Film,” Journal of the Society of Photographic Science and Technology of Japan, 2006, 69, 4, 271-275). Methylene blue is crystalline powder with a blue luster and is soluble in water or ethanol. Therefore, methylene blue is used in a solution state as a dye or an indicator. Then, methylene blue reacts with active oxygen to undergo decomposition and loses its blue color. Therefore, the presence or absence of active oxygen in an induced flow is confirmable by the decolorization (loss of blue color) of methylene blue.

Specifically, the following operation was conducted.

Methylene blue (produced by KANTO CHEMICAL CO., INC., highest quality) was mixed with distilled water to prepare a 0.01 mass % methylene blue solution. 15 mL of the methylene blue solution was placed into a petri dish (AB4000, manufactured by EIKEN CHEMICAL CO., LTD., a column shape having a diameter of 88 mm). The ultraviolet lamp was arranged on the petri dish so that the distance between the liquid surface of the methylene blue solution in the petri dish and the ultraviolet lamp became 6 mm. Note that in order to establish the relationship between the opening part and the petri dish and the liquid surface, a device configuration position was adjusted where necessary so as not to affect this confirmation test.

Next, the ultraviolet lamp was lit up, while applying an AC voltage with a sine waveform having an amplitude of 2.4 kVpp and a frequency of 80 kHz between both electrodes of the plasma actuators, thereby supplying an induced flow flowing out from the opening part toward the liquid surface for 30 μminutes. Note that the ultraviolet lamp was adjusted so that illumination measured at the exposed surfaces of the dielectrics of the plasma actuators on the side opposite to the ultraviolet lamp was 1370 μW/cm² without powering on the plasma actuators.

The methylene blue solution after the supply of the induced flow was transferred into a cell, and a change in the light absorption amount of methylene blue was measured using a spectrophotometer (V-570 manufactured by JASCO Corporation). Since methylene blue exhibits strong absorption at a wavelength of 664 nm, the degree of the decolorization of methylene blue is calculatable from a change in absorbance at the wavelength. In this test, when only distilled water was first placed into the reference cell and a 0.01% methylene blue solution before the supply of the induced flow was placed into the sample cell and measured, the absorbance was 2.32 Abs. On the other hand, the absorbance of the methylene blue solution after the supply of the induced flow was 0.21 Abs. Therefore, the reduction rate of the absorbance was 91% (((2.32−0.21)/2.32)×100).

### 3-2. Treatment (Sterilization) Test

Using the gas treatment device 108, a sterilization test for E. coli was conducted in the following procedure. Note that all the equipment used in this sterilization test were those having undergone high-pressure steam sterilization using an autoclave. Further, this sterilization test was conducted inside of a clean bench.

First, E. coli (product name “KWIK-STIK (E. coli (Escherichia coli) ATCC8739)”, produced by Microbiologics, Inc.) was placed into an Erlenmeyer flask containing an LB medium (prepared by dissolving 200 mL of distilled water in a mixture of 2 g of tryptone (product name “Bacto Tryptone,” produced by Life Technologies Japan Ltd.), 1 g of yeast extract (product name “Yeast Extract,” produced by Life Technologies Japan Ltd.), and 1 g of sodium chloride (product name “sodium chloride, highest quality,” produced by Kishida Chemical Co., Ltd.)). Subsequently, the Erlenmeyer flask was shaken and incubated using an incubator shaker (TA-25R-3F, manufactured by Takasaki Kagaku Kikai Co. Ltd.,) for 48 hours at 37° C. and at 80 rpm to obtain an E. coli solution. A viable cell count of the obtained E. coli solution was 9.2×10⁹ (CFU/mL).

20 mL of the produced E. coli solution was placed into a petri dish, and the petri dish was arranged on the bottom surface of the fungus-containing-gas preparation unit 407 to be set as an E. coli solution petri dish 414. As a method for generating gas containing E. coli, a method for forming mist of the coli solution in the fungus-containing-gas preparation unit 407 was employed. That is, a method for forming mist of the coli solution using an ultrasonic sprayer (manufactured by REN HE Company) was employed. The ultrasonic sprayer includes a substrate (not shown) to perform control and a vibrator 413 connected to the substrate by a wire. The vibrator is composed of ABS resin, silicone resin, or ceramics material, and the ceramics material has a circular sheet shape having a diameter of 16 mm and is equipped with numerous minute holes measuring 5 μm in size from its upper surface to lower surface. Since the ceramic material has piezoelectric properties, ultrasonic vibration is generated in the vibrator by the application of a voltage. When ultrasonic waves are generated with the lower surface of the vibrator coming into contact with the liquid surface of the coli solution, the coli solution forms fine liquid droplets as it passes through the minute holes so as to be absorbed, and mist is ejected from the upper surface of the vibrator.

The suction pump connected to the fungus collection unit 408 via the resin tube 409 is operated to create a negative pressure inside of the fungus collection unit 408, thereby enabling the transfer of the mist of the coli solution to the fungus collection unit 408. At this time, if a hole 415 is bored in the fungus-containing-gas preparation unit 407, external air is taken in the fungus-containing-gas preparation unit 407 from the hole 415, enabling the transfer of the mist of the coli solution.

In the fungus collection unit 408, a stamp medium 410 (Petan Check 25 PT1025, produced by EIKEN CHEMICAL CO., LTD.) was provided at its bottom.

Subsequently, the ultrasonic sprayer, the suction pump, and the active oxygen supply device were operated at the same time, and this time point was set as 0 seconds. A 5 V DC voltage was applied to the ultrasonic sprayer. The suction amount of the suction pump was set at 2.5 L/min. The active oxygen supply device 101 was operated, applying a voltage with a sine waveform having an amplitude of 2.4 kV and a frequency of 80 kHz to the plasma actuators. Then, ultraviolet rays were irradiated from the ultraviolet lamp to achieve an illumination of 1370 μW/cm² μmeasured without powering on the plasma actuators. In this manner, fungus-containing-gas passing through the gas flow path was treated using an induced flow containing active oxygen. Note that the voltages applied to the plasma actuators and the ultraviolet light source of the active oxygen supply device were set at the same values as in the confirmation test for active oxygen in section 3-1. This test was conducted for 15 seconds and completed when the pump, the active oxygen supply device, and the ultrasonic sprayer were stopped at the same time. The flow velocity of gas in this test was 0.46 m/sec.

After the test was completed, the stamp medium of the fungus collection unit 408 was placed into a constant temperature bath (product name: IS600, manufactured by Yamato Scientific Co., Ltd.) and incubated for 24 hours at 37° C. to obtain a sample No. 1. The number of generated colonies was counted to obtain a viable cell count after the sterilization treatment. As a result, the viable cell count associated with the sample No. 1 was 0 (CFU).

Next, except that the treatment by the active oxygen supply device was not conducted, an incubation test was conducted as in the case of the sample No. 1 to obtain a sample No. C1, and the number of colonies was counted to calculate a viable cell count. As a result, the viable cell count associated with the sample No. C1 was 197 (CFU).

Accordingly, the sterilization rate of E. coli by the active oxygen supply device according to this test was 100.0% (=(197−0)/197).

### Examples 2 to 5

Gas treatment devices were manufactured in the same manner as Example 1 except that the illumination and wavelengths of ultraviolet rays, as well as the thicknesses and materials of the dielectrics of plasma actuators, were changed as shown in Table 1. The gas treatment devices were then evaluated to provide Examples 2 to 5. The evaluation results are shown in Table 1. Note that an ultraviolet LED (having a peak wavelength of 280 nm) was used as an ultraviolet light source in Example 5.

### Example 6

A gas treatment device was manufactured in the same manner as Example 1 except that the height of a gas flow path was changed as shown in Table 1. The gas treatment device was then evaluated to provide Example 6. The evaluation results are shown in Table 1. Note that in Example 6, the distance between the upper end of a petri dish and an opening part was set at 10 mm in the confirmation test for active oxygen in section 3-1. This is to accommodate a change in the height of the gas flow path, which corresponds to the conditions for conducting a treatment (sterilization) test in the section 3-2.

The flow path for fungus-containing-gas was expanded, resulting in an increased distance from the opening part. However, gas sterilization treatment performance remained sufficient.

### Comparative Examples 1 to 3

The conditions for Comparative Examples 1 to 3 were the same as those for Example 1, except for the following differences.

Comparative Example 1: No voltage was applied to plasma actuators, and no ultraviolet rays were irradiated.

Comparative Example 2: A voltage was applied to plasma actuators, and no ultraviolet rays were irradiated.

Comparative Example 3: No voltage was applied to plasma actuators, and ultraviolet rays were irradiated.

TABLE 1

| | | | | | | | | | Reduction | | |
|---|---|---|---|---|---|---|---|---|---|---|---|
| | | | | Thickness | | | | | rate of | | |
| | | Peak | Material | of | Width of | Wind | | | absorbance | The | |
| | | wavelength | of | dielectric | gas flow | velocity | Ozone | UV | of methylene | number of | Sterilization |
| | | of UV | dielectric | of PA | path | of gas | concentration | illumination | blue solution | colonies | rate |
| | | (nm) | of PA | (μm) | (mm) | (m/sec) | (μg/min) | (μW/cm²) | (%) | (CFU) | (%) |
| Examples | 1 | 254 | Glass | 150 | 6 | 0.46 | 39 | 1370 | 91 | 0 | 100.00 |
| | 2 | 254 | Glass | 150 | 6 | 0.46 | 39 | 1070 | 86 | 4 | 97.97 |
| | 3 | 254 | Glass | 250 | 6 | 0.46 | 32 | 1370 | 88 | 2 | 98.98 |
| | 4 | 254 | Polyimide | 150 | 6 | 0.46 | 38 | 1370 | 90 | 0 | 100.00 |
| | 5 | 280 | Glass | 150 | 6 | 0.46 | 39 | 1370 | 82 | 14 | 92.89 |
| | 6 | 254 | Glass | 150 | 12 | 0.46 | 39 | 1370 | 78 | 16 | 91.88 |
| Comparative | 1 | — | Glass | 150 | 6 | 0.46 | 0 | 0 | 0 | 197 | 0.00 |
| Examples | 2 | — | Glass | 150 | 6 | 0.46 | 39 | 0 | 43 | 105 | 46.70 |
| | 3 | 254 | Glass | 150 | 6 | 0.46 | 0 | 1370 | 3 | 90 | 54.31 |

In the table, PA represents a plasma actuator, and UV represents ultraviolet rays. Further, ozone concentration represents ozone concentration in a case where an ultraviolet light source was not powered on, and UV illumination represents the illumination of ultraviolet rays in a case where only the ultraviolet light source was operated.

As shown in Comparative Example 3, the decolorization of methylene blue did not occur due to ultraviolet rays. Further, as shown in Comparative Example 2, the slight decolorization of methylene blue was observed in a case where ozone was generated. Moreover, in a case where both the generation of ozone and the irradiation of ultraviolet rays were performed, the decolorization of methylene blue further advanced due to the high reactivity of active oxygen.

In Comparative Example 1, neither the plasma actuator nor active oxygen was not operated, resulting in no sterilization effect by ultraviolet rays, ozone, and active oxygen. In Comparative Example 2, a sterilization effect by ozone was observed to some degree, but it was not as effective as in Examples 1 to 6. It is presumed that the slight decolorization of methylene blue observed in Comparative Example 2 was not caused by active oxygen but was caused by ozone. In Comparative Example 3, a sterilization effect by ultraviolet rays was observed to some degree, but it was not as effective as Examples 1 to 6 and was insufficient as a gas treatment device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A gas treatment device comprising:
an active oxygen supply device; and
a gas flow path, wherein
the active oxygen supply device comprises
a housing having at least one opening part,
a plasma actuator arranged inside of the housing, and
an ozone decomposition device,
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the ozone decomposition device decomposes the ozone contained in the induced flow to generate active oxygen in the induced flow, and the induced flow results in an induced flow containing the active oxygen,
the plasma actuator and the ozone decomposition device are arranged so that the induced flow containing the active oxygen flows to an outside of the housing from the opening part, and
the active oxygen supply device is arranged so that the induced flow containing the active oxygen is supplied to the gas flow path from the opening part.

2. The gas treatment device according to claim 1, wherein,
when a cross section of the plasma actuator in a thickness direction is viewed,
the first electrode and the second electrode are arranged diagonally opposite to each other via the dielectric in the thickness direction of the plasma actuator, and
the first electrode is provided to coat a part of the first surface of the dielectric,
the first surface has an exposed part not coated with the first electrode,
at least a part of the exposed part and the second electrode overlap each other when the plasma actuator is viewed through from a side of the first electrode, and
the induced flow blows out along the exposed part of the dielectric overlapping the second electrode from an edge part of the first electrode on a side of the first direction in the cross section in the thickness direction.

3. The gas treatment device according to claim 1, wherein
an ozone generation amount per unit time in a state in which the ozone in the induced flow is not decomposed by the ozone decomposition device is at least 15 g/min in the plasma actuator.

4. The gas treatment device according to claim 1, wherein
the induced flow containing the active oxygen flowing to the outside of the housing from the opening part has a vector representing a blowing-out direction thereof, and the vector has a component parallel to a flowing direction of gas in the gas flow path.

5. The gas treatment device according to claim 1, wherein
a distance between the ozone decomposition device and the plasma generation device is not more than 10 mm.

6. The gas treatment device according to claim 1, wherein
the ozone decomposition device is arranged to be capable of treating gas passing through the gas flow path, via the opening part.

7. The gas treatment device according to claim 1, wherein
a width of the gas flow path is not more than 12 mm.

8. The gas treatment device according to claim 1, wherein
the ozone decomposition device is at least one device selected from a group consisting of
an ultraviolet light source irradiating the induced flow with ultraviolet rays to generate the active oxygen in the induced flow,
a heating device heating the induced flow to generate the active oxygen in the induced flow, and
a humidifying device humidifying the induced flow to generate the active oxygen in the induced flow.

9. The gas treatment device according to claim 8, wherein
the ozone decomposition device is the ultraviolet light source, and
a peak wavelength of ultraviolet rays emitted from the ultraviolet light source is 220 to 310 nm.

10. The gas treatment device according to claim 8, wherein
the ozone decomposition device is the ultraviolet light source, and
illumination of ultraviolet rays in the opening part is at least 40 μW/cm².

11. A treatment method for treating gas using active oxygen, the method comprising:
a step of preparing a gas treatment device comprising an active oxygen supply device and a gas flow path, wherein
the active oxygen supply device comprises
a housing having at least one opening part,
a plasma actuator arranged inside of the housing, and
an ozone decomposition device,
the plasma actuator comprises a first electrode, a dielectric, and a second electrode laminated together in this order,
the first electrode is an exposed electrode provided on a first surface representing one surface of the dielectric,
when a voltage is applied between the first electrode and the second electrode, the plasma actuator generates a dielectric barrier discharge oriented from the first electrode toward the second electrode, and blows out an induced flow containing ozone in a first direction representing one direction along a surface of the dielectric from the first electrode,
the ozone decomposition device decomposes the ozone contained in the induced flow to generate active oxygen in the induced flows, and the induced flow results in an induced flow containing the active oxygen, and
the plasma actuator and the ozone decomposition device are arranged so that the induced flow containing the active oxygen flows to an outside of the housing from the opening part, and
the active oxygen supply device is arranged so that the induced flow containing the active oxygen is supplied to the gas flow path from the opening part.

12. The gas treatment method according to claim 11, wherein
a width of the gas flow path is not more than 12 mm.
